(19) United States
(12) Patent Application Publication
(10) Pub. No.: US 2014/0296641 A1
(43) Pub. Date: Oct. 2, 2014

(54) COMPOSITIONS, FORMULATIONS AND METHODS OF BIO-BALANCING THE pH OF STERILE HYPOTONIC, ISOTONIC SALINE AND HYPERTONIC SALINE SOLUTIONS

(71) Applicants: William Randolph Warner, Punta Gorda, FL (US); Werner Gutmann, Powhatan, VA (US)

(72) Inventors: William Randolph Warner, Punta Gorda, FL (US); Werner Gutmann, Powhatan, VA (US)

(21) Appl. No.: 14/226,764

(22) Filed: Mar. 26, 2014

Related U.S. Application Data
(63) Continuation-in-part of application No. PCT/US13/43472, filed on May 30, 2013.
(60) Provisional application No. 61/806,307, filed on Mar. 28, 2013.

(51) Int. Cl. A61K 47/08; A61B 1/015; A61B 1/267; A61K 33/14; A61M 16/14

(52) U.S. Cl. CPC A61K 47/08; A61K 33/14; A61M 16/14; A61B 1/2676; A61B 1/015
USPC 600/158; 424/680; 128/200.14; 600/156

(57) ABSTRACT

Compositions, formulations and methods bio-balance the pH of sterile, sodium chloride inhalation and pulmonary irrigation solutions, such that the solutions are bio-similar to the homeostatic pH state of the Human Airway Surface Liquid (ASL) at about 7.4. These solutions are sterile, non-pyrogenic, additive and preservative free, and provided in sterile unit-of-use, blow-fill-seal vials. These solutions are intended for use in the induction of sputum production where sputum production is indicated, such as with Cystic Fibrosis patients and Bronchoalveolar lavage procedures. The solutions can be provided in concentrations from about 0.045% to about 10% sodium chloride in USP/Sterile water. The sodium chloride solutions are homogenous mixtures that include sterile water, sodium chloride, and sodium bicarbonate as a buffer. The solutions are provided in saline concentrations from 0.45% to 10%, for example, concentrations of 0.9%, 3%, 3.5%, 6%, and 7% with a pH of 7.4.

### CROSS-REFERENCE TO RELATED APPLICATION

This application is a continuation-in-part of PCT/US13/43472, filed May 30, 2013, and also claims the benefit of priority of U.S. provisional application No. 61/806,307, filed Mar. 28, 2013, the contents of each are herein incorporated by reference.

### BACKGROUND OF THE INVENTION

The present invention relates to compositions, formulations and methods of bio-balancing the pH of sterile, sodium chloride inhalation and pulmonary irrigation solutions, that are bio-similar to the homeostatic pH state of the Human Airway Surface Liquid (ASL) at about 7.4. These solutions are sterile, non-pyrogenic, additive and preservative free, and are provided in, for example, sterile unit-of-use, blow-fill-seal vials or sterile, unit-of-use syringes. These solutions are intended for use in the induction of sputum production where sputum production is indicated, e.g., as used by Cystic Fibrosis patients (CF) and/or in Bronchoalveolar Lavage (BAL) procedures.

In healthy subjects, the average Human Blood and Airway Surface Liquid pH is between 7.38 and 7.42 making it slightly alkaline. The alveolar blood/gas, exchange/profusion and innate immune system can be adversely affected with lower, acidic pH.

Saline (also referred to as saline solution) is a general term referring to a sterile solution of sodium chloride in water. It is used for intravenous infusion, rinsing contact lenses, nasal irrigation and inhaled forms. Physiological saline contains 0.9% of sodium chloride in water and is isotonic (i.e. having same osmotic pressure as blood serum).

Hypertonic saline solutions in concentrations greater than about 1% have shown to be bacteriocidal and bacteriostatic. In general, if an antibacterial agent is bacteriostatic, it means that the agent essentially stops bacterial cell growth (but does not kill the bacteria); if the agent is bacteriocidal, it means that the agent kills the bacterial cell (and may stop growth before killing the bacteria).

Alone, hypertonic sodium chloride solutions in concentrations greater than 1% have been shown to be bactericidal and bacteriostatic. The hypertonic sodium chloride solution's osmotic property creates mucocillary mobilization or motility of bacterial and fungal pathogens that harbor in pulmonary mucus. The solute concentration causes antimicrobial activity by diffusing water out of the cells. The osmotic property creates mucocillary mobilization by the thinning of the mucus, liquidization or lowering the viscosity of the mucus. The osmotic active properties of hypertonic saline creates diffusion of H₂O molecules. Fluids move fluids within the inflamed mucosa to the greater solute of the “higher concentration” (hypertonic) therefore, enhancing mucocillary clearance.

The United States Pharmacopeia (USP) has a pH guidance for Sodium Chloride Inhalation Solutions, specified as having a pH between 4.5 and 7.0. Of three 7% saline solutions currently cleared by the Center for Devices and Radiological Health (CDRH) that are in commerce, they have an average pH of 5.92, clearly in the acidic spectrum.

The pH of a solution is a measure of the molar concentration of hydrogen ions in the solution and, as such, is a measure of the acidity or basicity of the solution. The letters pH stand for “power of hydrogen” and numerical value for pH is just the negative of the power of 10 of the molar concentration of H⁺ ions. pH=−log₁₀[H⁺]. The usual range of pH values encountered is between 0 and 14, with 0 being the value for concentrated hydrochloric acid, 7 the value for pure water (neutral pH), and 14 being the value for concentrated sodium hydroxide.

An important example of pH is that of the blood. The nominal value for blood pH of 7.4 is regulated very accurately by the body. If the pH of the blood gets outside the range from 7.3 to 7.5, the results can be serious and even fatal.

A pH of less than 7 may provide a more favorable acidic environment for pulmonary bacterial colonization. Therefore, the currently cleared sodium chloride inhalation liquids, with an average pH of 5.92, may prove detrimental to pulmonary bacterial colonization.

As can be seen, there is a need for sterile, sodium chloride inhalation and pulmonary irrigation solutions, that are bio-similar to the homeostatic pH state of the Human Airway Surface Liquid (ASL) at 7.4.

### SUMMARY OF THE INVENTION

In one aspect of the present invention, a sodium chloride inhalation and pulmonary irrigation solution comprises an aqueous solution of sodium chloride having a concentration from about 0.45% percent to about 10 percent; and a buffer to maintain a pH of about 7.4.

In another aspect of the present invention, a method for bio-balancing the pH of sterile sodium chloride inhalation and pulmonary irrigation solutions, comprises adjusting the pH of the solutions to be bio-similar to the homeostatic pH state of the human airway surface liquid.

These and other features, aspects and advantages of the present invention will become better understood with reference to the following drawings, description and claims.

### BRIEF DESCRIPTION OF THE DRAWINGS

FIG. 1A is a front view of a hexagonal shaped, sterile, blow-fill-seal vial used for sterile sodium chloride solution for inhalation and BAL irrigation;

FIG. 1B is a side view of the hexagonal shaped vial of FIG. 1A;

FIG. 1C is a front view of a plurality of hexagonal shaped vials, removably attached together;

FIG. 2A is a front view of a round shaped, sterile, blow-fill-seal vial used for sterile sodium chloride solution for inhalation and BAL irrigation;

FIG. 2B is a side view of the round shaped vial of FIG. 2A;

FIG. 2C is a front view of a plurality of round shaped vials, removably attached together;

FIG. 3A is an exploded side view of a pre-filled syringe used for sterile sodium chloride solution for pulmonary lavage or irrigation;

FIG. 3B is a perspective view of the syringe of FIG. 3A attached to delivery tubing;

FIG. 3C is a perspective view of the syringe of FIG. 3A being attached directly to a working port of a bronchoscope; and

FIG. 4 is an exploded perspective view of a nebulizer used with the vials of FIGS. 1A through 2C.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides compositions, formulations and methods of bio-balancing the pH of sterile, sodium chloride inhalation and pulmonary irrigation solutions, such that the solutions are bio-similar to the homeostatic pH state of the Human Airway Surface Liquid (ASL) at about 7.4. These solutions are sterile, non-pyrogenic, additive and preservative free, and provided in sterile unit-of-use, blow-fill-seal vials. These solutions are intended for use in the induction of sputum production where sputum production is indicated, such as with Cystic Fibrosis patients and Bronchoalveolar lavage procedures. The solutions can be provided in concentrations from about 0.45% to about 10% sodium chloride in USP/Sterile water. The sodium chloride solutions are homogenous mixtures (complete solutions) that include sterile water, sodium chloride, and sodium bicarbonate as a buffer. The solutions are provided in saline concentrations from 0.45% to 10%, for example, concentrations of 0.9%, 3%, 3.5%, 6%, 7% with a pH of 7.4.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying drawings and detailed in the following description.

It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law.

Accordingly, definitions are provided where certain terms related to the invention are defined specifically for clarity, but all of the definitions are consistent with how a skilled artisan would understand these terms. Particular methods, devices, and materials are described, although any methods and materials similar or equivalent to those described herein can be used in the practice or testing of the invention. All references referred to herein are incorporated by reference herein in their entirety.

As used herein, the term “saline” refers to salt, and more particularly, to sodium chloride.

As used herein, the term “hypertonic” refers to a solution with a solute concentration that is higher than that inside cells present in that solution, and therefore causes water to diffuse out of the cells. The term “hypertonic” is a relational term expressing the greater relative solute concentration of one solution compared with another (i.e., the latter is “hypertonic” to the former). A hypertonic solution has a lower water potential than a solution that is hypotonic to it and has a correspondingly greater osmotic pressure.

As used herein, the term “osmotic activity” refers to the net diffusion of water across a selective permeable membrane that is permeable in both directions to water, but varying permeable to solutes, wherein the water diffuses from one solution into another of lower water potential.

By “pharmaceutically acceptable” is meant a material or materials that are suitable and approved FDA/USP active ingredients, manufactured in accordance to cGMP compliance in a registered FDA facility and not biologically or otherwise undesirable, i.e., that may be administered to an individual along with an active agent or solution without causing any undesirable biological effects or interacting in a deleterious manner with any of the other components of the pharmaceutical formulation in which it is contained.

By the terms “effective amount or concentration” or “therapeutically effective amount” of an agent as provided herein are meant a nontoxic but sufficient amount of the agent to provide the desired therapeutic effect. The exact amount required will vary from subject to subject, depending on the age, weight, and general condition of the subject, the severity of the condition being treated, the judgment of the clinician, and the like. Thus, it is not possible to specify an exact “effective amount.” However, an appropriate “effective” amount in any individual case may be determined by one of ordinary skill in the art using only routine experimentation.

The term “bio-similar to the homeostatic pH state of the Human Airway Surface Liquid (ASL)” is the thin layer of liquid at the air-facing epithelial surface in the upper and lower airways with a pH of 7.4±. The regulation of ASL volume, ionic composition, and pH is believed to be important in normal airway physiology and in the pathophysiology of genetic and acquired diseases of the airways such as cystic fibrosis and asthma. Abnormalities of the ASL may induce bronchoconstriction, cough reflex and interfere with epithelial cell ionic homeostasis and airway defense mechanisms such as antimicrobial activity and bacterial clearance.

Cystic Fibrosis (CF) and Mechanically Ventilated patients are often compromised with respiratory acidosis as a result from a build-up of carbon dioxide (CO₂) in the blood (hypercapnia) due to hypoventilation. More CO₂ translates into a lower pH (CO₂+H₂O----->HCO₃----->HCO₃⁻+H⁺).

Abnormally low pH of the ASL may facilitate bacterial survival in the airway lumen. Phagocytic cells are less efficient at ingesting and killing bacteria at lower extracellular pH. In addition, pseudomonas aeruginosa, the organism most typically associated with CF and Ventilator-Associated Pneumonia (VAP) patients.

The term “Bronchoalveolar lavage” (BAL) is a medical procedure in which a bronchoscope is passed through the mouth or nose into the lungs and sterile 0.9% sodium chloride solution is squirted into a small part of the lung and then recollected for microbiological examination. BAL is typically performed to diagnose lung disease. In particular, BAL is commonly used to diagnose infections in people with immune system problems, pneumonia in people on ventilators.

Ventilator-Associated Pneumonia (VAP) is a leading cause of morbidity and mortality in the Intensive Care Unit (ICU). The incidence of VAP varies greatly, ranging from 6 to 52% of intubated patients, depending on patient risk factors. Attributable mortality approaches 50% when VAP is caused by the more virulent organisms that typify late-onset VAP, occurring 4 or more days into mechanical ventilation. VAP is commonly caused by antibiotic-resistant nosocomial organisms (e.g., Pseudomonas aeruginosa).

Community-Acquired Pneumonia (CAP) can be caused by any microorganism that can cause VAP, however there are several bacteria which are particularly important causes of VAP because of their resistance to commonly used antibiotics. These bacteria are referred to as Multi-Drug Resistant (MDR). Pseudomonas aeruginosa is the most common MDR gram-negative bacterium causing VAP. Pseudomonas has natural resistance to many antibiotics.

The present invention relates to compositions, formulations and methods bio-balance the pH of sterile, sodium chloride inhalation and pulmonary irrigation solutions, such that the solutions are bio-similar to the homeostatic pH state of the Human Airway Surface Liquid (ASL) at 7.4.

The compositions of the present invention are sterile, non-pyrogenic, additive and preservative free, and provided in sterile unit-of-use, blow-fill-seal vials. These compositions are intended for use in the induction of sputum production where sputum production is indicated, such as with Cystic Fibrosis patients and Bronchoalveolar lavage procedures.

The compositions of the present invention can be provided in concentrations from about 1.0 percent to about 10 percent sodium chloride in USP/sterile water. The sodium chloride solutions are homogenous mixtures (complete solutions) that include sterile water, sodium chloride, and a buffer. Typically, sodium bicarbonate can be used as the buffer. The sodium bicarbonate can be disposed in a concentration from about 0.03 to about 0.05 mg/ml, typically about 0.04 mg/ml. The compositions are provided in saline concentrations from 0.45% to 10%, for example, concentrations of 0.9%, 3%, 3.5%, 6%, 7% with a pH of 7.4.

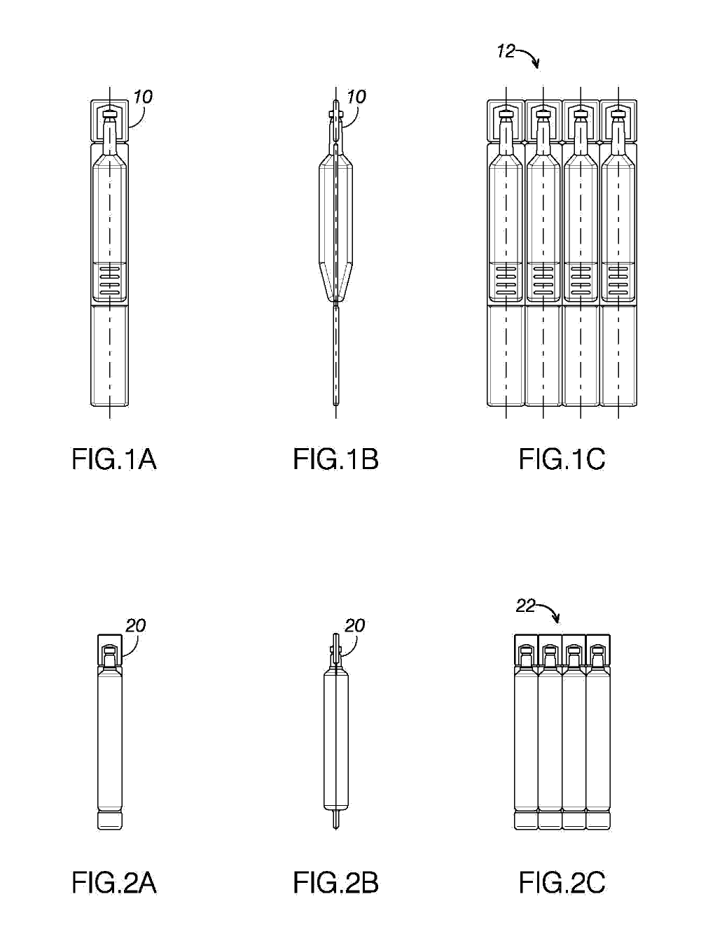

Referring to FIGS. 1A through 1C, in some embodiments, the solutions for inhalation can be provided in blow-fill-seal vials 10. The vials 10 can be provided as a package 12 of several vials 10 removably interconnected. The vials 10 can contain the sodium chloride solution as described above.

Referring to FIGS. 2A through 2C, in some embodiments, the solutions for inhalation can be provided in round-shaped vials 20. The vials 20 can be provided as a package 22 of several vials 20 removably interconnected. The vials 20 can contain the sodium chloride solution as described above.

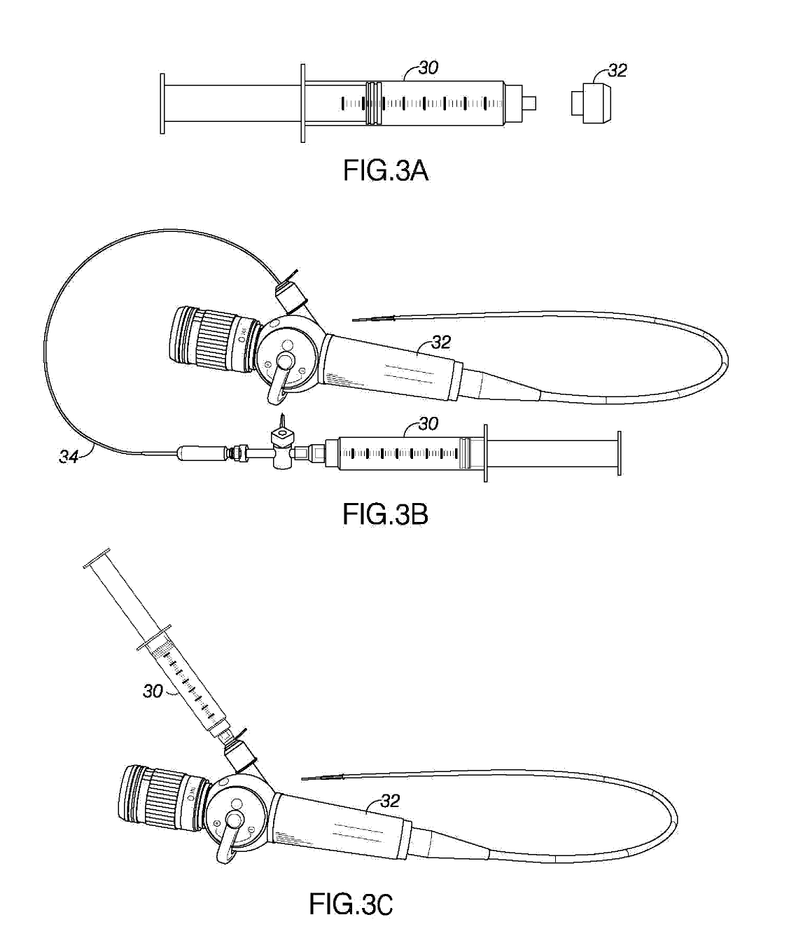

Referring to FIGS. 3A through 3C, a syringe 30 may be provided with the sodium chloride solutions described above. The syringes 30 may be sterile, pre-packaged syringes having a removable, sealing cap 32. The cap 32 may be removed during use and a flexible tube 34 attached for delivering the solution from the syringe 30 to a bronchoscope 34. In some embodiments, the syringe 30 may be attached directly to the inlet port of the bronchoscope 34, as shown in FIG. 3C. The bronchoscope 34 may be used, for example, with suction for pulmonary lavage or irrigation. The syringes 30 may be from about 10 to about 30 ml in size.

In one embodiment, the syringes 30 can be pre-filled with 20 ml of sodium chloride solution.

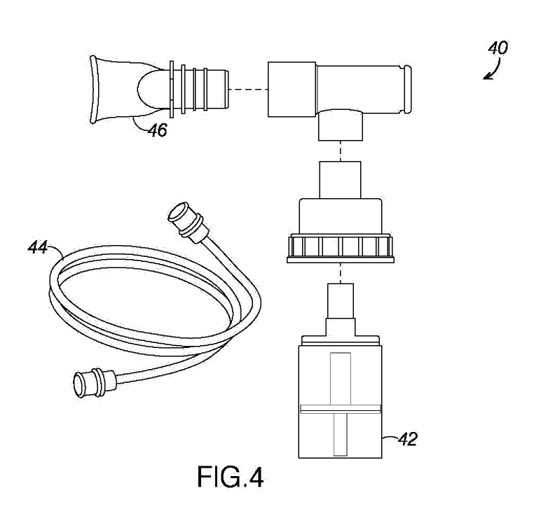

Referring now to FIG. 4, the vials 10, 20 described above may be used with a nebulizer 40. The nebulizer may be a standard nebulizer as is known in the art. The user can open the vial 10, 20 and pour the contents into a reservoir 42 of the nebulizer 40. Supplemental air (or oxygen-enriched air, or pure oxygen, for example) can be delivered to the nebulizer 40 via tubing 44 and the user can breathe through the mouthpiece 46 for delivery of the sodium chloride solution to their lungs. The nebulizer can be, for example, an aerolizing nebulizer, such as a pneumatic, vibrating mesh or ultrasonic delivery device. The vials 10, 20 can be, for example, filled with about 4 ml of sodium chloride solution for delivery to the reservoir of the nebulizer.

It should be understood, of course, that the foregoing relates to exemplary embodiments of the invention and that modifications may be made without departing from the spirit and scope of the invention as set forth in the following claims.

What is claimed is:

1. A sodium chloride inhalation and pulmonary irrigation solution comprising:
an aqueous solution of sodium chloride having a concentration from about 0.45% percent to about 10 percent;
a buffer to maintain a pH of about 7.4.

2. The sodium chloride inhalation and pulmonary irrigation solution of claim 1, wherein the buffer is sodium bicarbonate.

3. The sodium chloride inhalation and pulmonary irrigation solution of claim 1, wherein the sodium chloride concentration is selected from the group consisting of 9%, 3%, 3.5%, 6% and 7%.

4. The sodium chloride inhalation and pulmonary irrigation solution of claim 1, wherein the sodium chloride solution is provided in a sterile, sealed vial.

5. The sodium chloride inhalation and pulmonary irrigation solution of claim 4, wherein the sterile, sealed vial contains an appropriate volume for use in a nebulizer device.

6. The sodium chloride inhalation and pulmonary irrigation solution of claim 1, wherein the sodium chloride solution is provided in a sterile, sealed syringe.

7. The sodium chloride inhalation and pulmonary irrigation solution of claim 6, wherein the syringe contains an appropriate volume for use in a bronchoscope device.

8. A method for bio-balancing the pH of sterile sodium chloride inhalation and pulmonary irrigation solutions, comprising adjusting the pH of the solutions to be bio-similar to the homeostatic pH state of the human airway surface liquid.

9. The method of claim 8, wherein the homeostatic pH state is a pH of about 7.4.

10. The method of claim 8, wherein the sterile sodium chloride inhalation and pulmonary irrigation solutions are pH adjusted with a buffer.

11. The method of claim 10, wherein the buffer includes sodium bicarbonate.

12. A medical device co-interfaced with a sodium chloride inhalation and pulmonary irrigation solution, the sodium chloride inhalation and pulmonary irrigation solution comprising:
an aqueous solution of sodium chloride having a concentration from about 0.45% percent to about 10 percent;
a buffer to maintain a pH of about 7.4

13. The medical device of claim 12, wherein the medical device is selected from the group consisting of a nebulizer and a bronchoscope.

14. The medical device of claim 12, wherein the medical device is a nebulizer.

15. The medical device of claim 14, wherein the sodium chloride inhalation and pulmonary irrigation solution is provided in a sterile, sealed vial.

16. The medical device of claim 15, wherein the sterile, sealed vial contains an appropriate volume for use in a nebulizer.

17. The medical device of claim 12, wherein the medical device is a bronchoscope.

18. The medical device of claim 17, wherein the sodium chloride inhalation and pulmonary irrigation solution is provided in a sterile, sealed syringe.

19. The medical device of claim 18, wherein the syringe contains an appropriate volume for use in a bronchoscope device.

20. The medical device of claim 12, wherein the sodium chloride concentration is selected from the group consisting of 0.9%, 3%, 3.5%, 6% and 7%.
